(19) United States
(12) Patent Application Publication
(10) Pub. No.: US 2009/0325302 A1
(43) Pub. Date: Dec. 31, 2009

(54) Diagnostic marker for diabetic vascular complications

(75) Inventors: Ayad A. Jaffa, Mount Pleasant, SC (US); William R. Usinger, Lafayette, CA (US)

Correspondence Address: FIBROGEN, INC., 409 Illinois Street, San Francisco, CA, 94158, US

(73) Assignee: FibroGen, Inc., South San Francisco, CA (US)

(21) Appl. No.: 11/919,607

(22) PCT Filed: May 5, 2006
(86) PCT No.: PCT/US2006/017755

§ 371 Date: Sep. 14, 2009

Related U.S. Application Data
(60) Provisional application No. 60/678,251, filed on May 5, 2005.

(51) Int. Cl. G01N 33/68

(52) U.S. Cl. 436/86

(57) ABSTRACT

The present invention relates to the discovery that CTGF is a diagnostic marker indicative of increased risk for development and progression of vascular disease.

This application claims the benefit of U.S. Provisional Application Ser. No. 60/678,251, filed on 5 May 2005, which is incorporated by reference herein it its entirety.

### FIELD OF THE INVENTION

The present invention relates to the discovery that CTGF is a diagnostic marker indicative of increased risk for development and progression of vascular disease.

### BACKGROUND OF THE INVENTION

Diabetes mellitus is associated with increased morbidity and mortality derived mainly from cardiovascular complications. The progression of vascular lesions is enhanced in the diabetic state and this risk is greatly accentuated by the coexisting hypertension. (Christlieb et al (1981) Diabetes 30 (Suppl 2):90-96; Krolewski et al (1988) N Engl J Med 318:140-145.) The mechanisms by which diabetes and hypertension cosegregate and accelerate vascular damage are as yet undefined. Both conditions are associated with endothelial dysfunction, accumulation of inflammatory cells, vascular smooth muscle cell (VSMC) proliferation and migration, and extracellular matrix deposition in the vessel wall. (See Ross (1993) Nature 362:801-809; Clowes and Karnovsky (1977) Nature 265:625-626; Clowes et al (1983) Lab Invest 49:327-333; and Jackson and Schwartz (1992) Hypertension 20:713-736.)

The development of micro- and macro-albuminuria in diabetic and non-diabetic individuals augments risk for the development of macrovascular disease. Type 1 diabetic patients with proteinuria have a risk of macrovascular disease increased ten-fold relative to that of type 1 patients without proteinuria. The relation of microalbuminuria to vascular disease complications such as carotid intima-medial thickness (IMT) was recently illustrated in the DCCT/EDIC-cohort of type 1 diabetic patients (The Diabetes Control and Complications Trial/Epidemiology of Diabetes and Complications Research Group (2003) N Engl J Med 348:2294-2303). Diabetic renal disease is associated with elevations of blood pressure and dyslipidemia, conditions that typically precede and accelerate the progression of vascular disease in diabetic patients (Perkins et al (2003) N Engl J Med 348:2285-2293).

CTGF was originally identified as a product of human umbilical vein endothelial cells that was both chemotatic and mitogenic for fibroblasts (See, e.g., Bradham et al (1991) J Cell Biol 114:1285-1294 and U.S. Pat. No. 5,408,040). CTGF belongs to a gene family, CCN, named after prototype members of this family, CTGF, Cyr61, and Nov (Bork (1993) FEBS Lett 327:125-130). The molecular weight of CTGF-like factors varies between 35-40 kDa, and the structure of these molecules consists of four modules: an N-terminal IFGBP-like domain, a Von Willebrand factor type C repeat domain, a thrombospondin type 1 repeat domain, and a C-terminal dimerization domain (Bork (1993) FEBS Lett 327:125-130).

CTGF is characterized by 38 conserved cysteine residues that constitute over 11% of its total amino acid content. Cysteines encoded within each of the four exons of the secreted protein are internally paired leading to the creation of amino and carboxy-terminal domains joined by a short, flexible and protease-sensitive 32 amino acid peptide (Bork (1993) FEBS Lett 327:125-130). CTGF is readily cleaved within this so-called “hinge” region resulting in the amino terminal fragment of CTGF (CTGF N-fragment; see International Publication No. WO 00/035936), the predominant form of CTGF present in blood and urine.

As changes in the plasma level of CTGF N fragment are predictive of the degree of activation and production of CTGF, the present study was conducted to determine whether circulating levels of CTGF and CTGF N-fragment mark an increased risk for development of vascular and renal disease in type 1 diabetic patients. Therefore, the present invention provides a diagnostic marker indicative of increased risk for development and progression of vascular disease.

### BRIEF DESCRIPTION OF THE DRAWINGS

FIG. 1 shows cumulative distribution of logarithm (log) CTGF N-fragment by hypertensive status.

### SUMMARY OF THE INVENTION

The present invention provides a method for diagnosing a risk for development of a vascular complication associated with diabetes in a subject having or at risk for having diabetes, the method comprising obtaining a biological sample from the subject, measuring the level of CTGF or of CTGF fragment in that biological sample, and comparing the level of CTGF or of CTGF fragment in the biological sample to standard levels of CTGF or of CTGF fragment, where an elevated level of CTGF or of CTGF fragment in the biological sample is indicative of a risk for development of a vascular complication associated with diabetes.

Typically, the subject having or at risk for having diabetes is a human subject.

In some embodiments, the vascular complication is a macrovascular complication or a microvascular complication. In particular, the vascular complication may be a cardiovascular complication or a cerebrovascular complication; or a complication of the peripheral vasculature.

In some embodiments, the vascular complication is carotid intima-medial thickness.

Typically, the level of CTGF fragment or of CTGF in the biological sample is detectable and quantifiable using an assay described in International Publication No. WO 03/024308.

In some embodiments, the biological sample is a sample derived from bodily fluids. In particular, the biological sample is urine or plasma.

In preferred embodiments, the subject has type 1 diabetes. Such a subject may also have increased blood pressure or microalbuminuria.

### DESCRIPTION OF THE INVENTION

It is to be understood that the invention is not limited to the particular methodologies, protocols, cell lines, assays, and reagents described herein, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments, a reference to an “antibody” is a reference to one or more antibodies and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (introduction to Biotechniques Series), 2nd ed. (Newton & Graham eds., 1997, Springer Verlag).

The present invention relates to the discovery that CTGF is a diagnostic marker indicative of increase risk for development and progression of vascular disease.

In one aspect, the present invention provides a method for diagnosing a risk for development of a vascular complication associated with diabetes in a subject having or at risk for having diabetes, the method comprising obtaining a biological sample from the subject, measuring the level of CTGF or of CTGF fragment in that biological sample, and comparing the level of CTGF or of CTGF fragment in the biological sample to standard levels of CTGF or of CTGF fragment, where an elevated level of CTGF or of CTGF fragment in the biological sample is indicative of a risk for development of a vascular complication associated with diabetes.

In preferred embodiments of the present invention, the subject having or at risk for having diabetes is a human subject. Whether a subject has or is at risk for having diabetes can be determined by any measure accepted and utilized by those of skill in the art. For example, a human subject having a blood glucose level above about 200 mg/dL (e.g., as determined in a fasting blood glucose test, an oral glucose tolerance test, or a random blood glucose test) may be characterized as a subject having diabetes. Therefore, in certain aspects, it is contemplated that a human subject having a blood glucose level above about 200 mg/dL is a suitable subject for treatment with the methods of or use of medicaments provided by the present invention. A subject at risk for having diabetes, for example, a human subject at risk for having diabetes, can be identified by an assessment of one or more of various factors known to be associated with an increased risk of developing diabetes, including family history of diabetes, certain ethnic or racial groups, a history of gestational diabetes, obesity, in particular, high levels of visceral or abdominal fat, a sedentary lifestyle, age, high blood pressure, schizophrenia, etc., as well as altered glucose metabolism, including impaired glucose tolerance (IGT) or prediabetes.

In certain embodiments, the vascular complication is a macrovascular complication; in other embodiments, a microvascular complication. In various embodiments, the complication is selected from the group consisting of a cardiopathy, a nephropathy, a neuropathy, and a retinopathy. In one embodiment, the complication is a cardiovascular complication or a cerebrovascular complication. In another embodiment, the complication is a complication of the peripheral vasculature.

In preferred embodiments of the present methods, the measuring the level of CTGF fragment or of CTGF in the biological sample comprises detecting and quantitating levels of CTGF or of CTGF fragment using any of the various assays described in International Publication No. WO 03/024308, which reference is incorporated herein by reference in its entirety. (See, e.g., Example 5 in International Publication No. WO 03/024308.)

The biological sample is, in preferred aspects, a sample derived from bodily fluids, secretions, tissues, or cells, including, but not limited to, saliva, blood, urine, serum, plasma, vitreous, etc.

The relevance and significance of connective tissue growth factor (CTGF) as a diagnostic marker indicative of increased risk for the development of vascular complications in diabetic patients in a cross-sectional study was examined. Circulating (i.e., plasma) and urinary levels of CTGF and CTGF N-fragment in 1,050 type 1 diabetic patients from the Diabetes Control and Complications Trial/Epidemiology of Diabetes Interventions and Complications (DCCT/EDIC) Study cohort were studied. Hypertensive diabetic subjects were found to have significantly higher levels of plasma log CTGF N-fragment than were normotensive subjects (3±0.04 ng/ml vs. 3.21±0.03 ng/ml, P=0.0005). Regression analysis determined that CTGF N-fragment levels positively and significantly correlated with systolic blood pressure as continuous variables (P<0.0001). Univariate and multivariate regression analysis showed a positive and independent association between CTGF N-fragment levels and log albumin excretion rate (AER) (P<0.0001). In categorical analysis, patients with macroalbuminuria had a significantly higher level of CTGF N-fragment than did microalbuminuric or normoalbuminuric diabetic subjects (P<0.0001). Univariate and multivariate regression analysis demonstrated that log CTGF N-fragment independently and significantly associated with the common carotid intima-media thickness (MT), a surrogate marker for macrovascular disease (<0.0428). Finally, the relative risk (RR) for increased carotid IMT was higher in patients with elevated levels of plasma CTGF N-fragment and macroalbuminuria than in patients with normal plasma CTGF N-fragment and normal albuminuria (RR=4.76; 95% confidence interval, 2.21-10.25, P<0.0001).

The present findings demonstrate that, in type 1 diabetic subjects, CTGF N-fragment levels are elevated in association with increased blood pressure; are independently correlated with AER and categorically elevated in patients with macroalbuminuria; are independently associated with carotid IMT; and are positively associated with greater IMT. Therefore, plasma CTGF levels are a risk marker of diabetic vascular disease.

The present findings show a positive and significant association between plasma CTGF levels and low density lipoprotein (LDL), demonstrating that LDL may modulate the levels of CTGF in diabetic patients. Further, previous reports have shown that the expression of CTGF in human aortic endothelial cells and mesangial cells is induced by LDL (Sohn et al (2005) Kidney Int 67:1286-1296). The induction of CTGF by LDL in mesangial cells was mediated via autocrine activation of TGF-β and via activation of c-Jun NH₂-terminal kinase (Sohn et al (2005) Kidney Int 67:1286-1296), suggesting that CTGF provides a pathway through which lipoproteins may promote vascular sclerosis in diabetes.

Microalbuminuria, a marker of diabetic nephropathy in type 1 diabetic patients, signifies high risk for progressive renal failure and disease. Microalbuminuria has been associated with increased cardiovascular mortality in populations of both diabetic and non-diabetic subjects and is also associated with generalized and glomerular endothelial dysfunction (Stehouwer et al (1992) Lancet 340:319-323). Identifying biomarkers that contribute to the development of microalbuminuria may provide insights into the mechanisms of diabetic vascular injury. As shown herein, CTGF N-fragment levels in plasma and urine of patients with macroalbuminuria were two-fold higher than levels in patients with microalbuminuria or with a normal albumin excretion rate. These findings suggest that CTGF is a marker for progressive nephropathy. The univariate and multivariate regression analyses revealed independent and positive associations between CTGF N-fragment and AER. These findings are in agreement with previous reports in the literature demonstrating an association between CTGF N-fragment and AER conducted in a much smaller number of type 1 diabetic patients. (See, e.g., International Publication

No. WO 03/024308.)

The present examples provide the first evidence of an association between CITGF N-fragment and elevated systolic and diastolic blood pressure. These data demonstrate that diabetic subjects with documented hypertension, irrespective of their current blood pressure or use of anti-hypertension medications, display a significantly higher level of plasma CTGF N-fragment than do normotensive diabetic subjects. This finding is of significance because risk for progressive renal injury and cardiovascular disease in diabetes is accentuated by hypertension. Interventional studies aimed at controlling blood pressure with ACE-inhibitors (ACEI) have been shown to significantly slow the development of diabetic renal injury (Lweis et al (1993) N Engl J Med 329:1456-1462). The beneficial effects conferred by ACEI therapy could be attributed to either a decrease in the conversion of angiotensin I to angiotensin II or to the decrease in the degradation of bradykinin (Gainer et al (1998) N Engl J Med 339:1285-1292).

A significant increase in the plasma levels of CTGF N-fragment in diabetic patients treated with ACEI has been shown. This increase in plasma CTGF levels in response to ACEI therapy may be attributed to the potentiation of bradykinin levels rather than to a decrease in angiotensin II formation. In this regard, the present studies demonstrate (data not shown) that bradykinin induces the expression of CTGF in human aortic endothelial cells as well as vascular smooth muscle cells and this regulation is mediated via autocrine activation of TGF-β.

The present invention further demonstrates an independent and positive association between plasma CTGF N-fragment levels and carotid and internal intima-media thickness, recognized markers for coronary as well as cerebral vascular disease in patients with type 1 diabetes (The Diabetes Control and Complications Trial/Epidemiology of Diabetes and Complications Research Group (2003) N Engl J Med 348:2294-2303). Given the association between hyperglycemia and hyperlipidemia with intima-media thickness and the influence of LDL and hyperglycemia on CTGF regulation, CTGF may be a mechanistic pathway through which lipoproteins and hyperglycemia mediate their deleterious effects on promoting vascular injury in diabetic patients.

In summary, the findings in the present study demonstrate that plasma CTGF N-fragment levels are an independent risk marker for vascular disease in patients with type 1 diabetes, and that CTGF serves as a disgnostic marker indicative of increased risk for development and progression of vascular disease.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Example 1

### Methods of the Present Study

The following methods were used in the current studies:

### Study Population

The study population was the North American DCCT/EDIC cohort, comprised of 1,325 type 1 diabetic patients from the original 1,441 DCCT subjects. The original DCCT (Diabetes Control and Complications Trial) cohort consisted of men and women between the ages of 1340 years with 1-15 years of diabetes at study entry (The Diabetes Control and Complications Trial Research Group (1993) N Engl J Med 329:977-986), enrolled between 1983 and 1989. Half of the patient population was randomly assigned to conventional diabetes treatment and the other half was assigned to intensive diabetes treatment. In 1993, the DCCT study was stopped after an average follow-up time of 6.5 years, when intensive treatment was clearly shown to reduce the risks of retinopathy, nephropathy, and neuropathy (The Diabetes Control and Complications Trial Research Group (1993) N Engl J Med 329:977-986). The patients were then invited to enroll in EDIC, a multicenter longitudinal observational study of the development of macrovascular complications and further progression of microvascular complications (EDIC Research Group (1999) Diabetes Care 22:99-111). At EDIC baseline in 1994, the average age of the DCCT/EDIC cohort was 35 years (range 19-50 years). Fifty four percent of the cohort were male, and the mean duration of the diabetes was 12±5 years. Fasting plasma samples were collected from the subjects for CTGF measurements and were shipped directly from participating EDIC clinics to Medical University of South Carolina (MUSC). The Institutional Review Boards of MUSC and all participating DCCT/EDIC clinics approved the study, and written informed consent was obtained from each patient participant.

### EDIC Procedures

On the approximate anniversary of enrolling the DCCT, each EDIC subject has a standardized annual history and physical examination, including a detailed evaluation of overall health, diabetes management, occurrence of diabetic complications, development of new disease, and medications used. Annual evaluations also included HbA1c, resting electrocardiograms, and arm blood pressure (BP) measurements. Blood pressure and HbA1c measurements were done at the same time of blood sampling. Blood pressure was measured in the right arm using a mercury column sphygmomanometer while the patient was in the sitting position. Renal function was assessed every second year and included measurements of the urinary albumin excretion rate (AER) in a standardized 4-h collection. (See The Diabetes Control and Complications Trial Research Group (1993) N Engl J Med 329:977-986.) Microalbuminuria was defined by the DCCT as an AER 40-299 mg/24 h, and macroalbuminuria as an AER≧300 mg/24 h. Normal albumin excretion was defined as an AER≦40 mg/24 h. About ninety eight percent of all AER measurements were carried out within one year of blood sampling.

### Ultrasonography and Image Analysis

Carotid ultrasonography was carried out in 1,325 patients (92% of the original DCCT cohort) as part of the EDIC baseline examination and was performed between June 1994 and April 1995 (2 years prior to measurements of CTGF) as previously described (Epidemiology of Diabetes Interventions and Complications (EDIC) Research Group (1999) Diabetes 48:383-390).

### CTGF Measurement

CTGF was measured in plasma and urine using ELISA assays previously described. (See, e.g., International Publication No. WO 03/024308, throughout the specification, which reference is incorporated herein by reference in its entirety.) (See Gilbert et al (2003) Diabetes Care 26:2632-2636.) Briefly, pairs of CTGF-specific monoclonal antibodies were used to construct two different ELISAs designed to capture and detect whole CTGF (W assay) or N-terminal CTGF fragments+whole CTGF (N+W assay). In the assay, microtiter plates (Maxisorb, Nunc, Rochester, N.Y.) were coated overnight at 4° C. with an anti-CTGF monoclonal antibody (10 μg/mL) used to capture CTGF, then were washed and blocked with PBS containing 1% BSA for at least 2 hours. A different non cross-blocking anti-CTGF mAb from that used as captur mAb, conjugated directly to alkaline phosphatase, was used for detection. Para-nitrophenol phosphate (PNPP) was used as the substrate for the colorimetric reaction. The plate was read at 405 nm (Vmax Plate Reader, Molecular Devices, Sunnyvale, Calif.). Standard curves were generated using rhCTGF standards run in triplicate with each set of samples. Samples were diluted 1:10 in assay buffer containing 50 μg/ml heparin, 0.1% Triton X-100, 0.1% BSA before being assayed in duplicate; CV on duplicates was within 15%. A quadratic fit to the standard curve was used for calibration. Spike-recovery experiments using recombinant human CTGF demonstrated quantitative detection in patient samples. Assay sensitivities (LLOQ) are 0.6 ng/ml for urine samples in the N+W assay and 5 ng/ml for serum samples in the W assay or N+W assay. The antibodies used in the ELISA are specific for CTGF, and do not cross-react with CCN family members cyr61 and nov. Although CTGF content in urine was measured using the N+W assay, the form of CTGF present in urine as measured by these assays was essentially N-fragment. Within-run % CVs were 5% and between-run % CVs were 15%.

### Statistical Analysis

The measured levels of CTGF in plasma and urine followed a skewed distribution and the Box-Cox transformation to CTGF was applied. The log transformation converted raw CTGF data to normality; thus, the log-transformed CTGF N-fragment was used in the analyses herein. In addition, logarithmic transformation of AER was used to provide normality of residuals. T tests were used to analyze continuous outcomes versus each covariate separately. Chi square tests were used to analyze discrete outcomes versus each covariate. Pearson's correlation coefficients as well as a Spearman nonparametric correlation were computed to assess the association between plasma log CTGF N-fragment and each of BP, AER, and IMT.

Plasma log CTGF N-fragment, log AER, and carotid IMT were all used as outcomes in regression analyses. In particular, when plasma log CTGF N-fragment was the outcome, ANOVA and ANCOVA were used to determine differences between mean levels of plasma log CTGF N-fragment in plasma and urine among nephropathy sub-groups. With outcomes log AER and carotid IMT, results from the univariable analyses were used to develop initial models for multiple linear regressions, to which backward model selection procedures were then applied to eliminate variables having non-significant partial tests and/or variables that were co-linear. Plasma log CTGF N-fragment was considered as a covariate in the regression models for both outcomes log AER and IMT. Log AER was considered as a covariate in the model for carotid IMT. These regression models were adjusted for other covariates such as age, HbA1c, duration of diabetes, gender, and DCCT treatment group. The square of the multiple partial correlation coefficient was calculated to estimate the increase in the percentage of the variance of the dependent variable explained by introducing that variable into a model that included all the other covariates. Bonferroni adjustment was performed for multiple comparisons. All statistical analyses were performed using SAS (v. 91). Measures of central tendency were expressed as mean±SD, wherein statistical significance was determined using a two-sided test and significance was assumed for p≦0.05.

### Example 2

### CTGF N-Fragment Levels in DCCT/EDIC-cohort of Type 1 Diabetic Patients

The clinical characteristics of the study population on which the CTGF measurements were performed are shown in Table 1 (clinical characteristics of the DCCT/EDIC-cohort by gender*). The circulating levels of plasma and the urinary excretion rate of CTGF N-fragment were measured in 1,052 type 1 diabetic patients. The relation of log CTGF N-fragment with biochemical parameters in the patient cohort is shown in Table 2 (univariate regression analyses to predict log CTGF N fragment levels). The univariable regression coefficient in Table 2 can be interpreted as the change in mean log CTGF N-fragment for one unit change in a given covariate. Another interpretation is exp (β) is approximately equal to the relative increase in CTGF N-fragment for one unit increase in a covariate.

TABLE 1

| | Male | | Female | | |
|---|---|---|---|---|---|
| Parameter | N | Mean ± SD | N | Mean ± SD | P |
| Age | 562 | 39.91 ± 6.74 | 452 | 39.09 ± 7.20 | 0.0632 |
| Weight (Kg) | 561 | 86.83 ± 14.84 | 451 | 72.52 ± 14.95 | 0.0001 |
| BMI (kg/m²) | 551 | 27.06 ± 3.86 | 439 | 26.34 ± 4.33 | 0.0055 |
| Waist-Hip-Ratio | 550 | 0.89 ± 0.06 | 438 | 0.77 ± 0.06 | 0.0001 |
| Duration of Diabetes (years) | 562 | 17.15 ± 4.66 | 452 | 17.69 ± 4.89 | 0.0743 |
| HbAlc (%) | 555 | 8.23 ± 1.31 | 447 | 8.22 ± 1.37 | 0.8643 |
| DCCT Group | 562 | 1.50 ± 0.50 | 452 | 1.47 ± 0.50 | 0.2870 |
| SBP(mmHg) | 551 | 122 ± 13.3 | 439 | 116 ± 13.82 | 0.0001 |
| DBP(rnmHg) | 551 | 77.09 ± 9.27 | 439 | 72.60 ± 9.01 | 0.0001 |
| MBP (mmHg) | 551 | 92.26 ± 9.61 | 439 | 87.08 ± 9.55 | 0.0001 |
| LDL(mg/dl) | 529 | 118.5 ± 31.19 | 423 | 110.0 ± 29.82 | 0.0001 |
| Total Cholesterol (mg/dl) | 533 | 189.7 ± 36.24 | 424 | 188.2 ± 33.75 | 0.5162 |
| Triglycerides (mg/dl) | 533 | 98.02 ± 70.88 | 424 | 77.21 ± 49.33 | 0.0001 |
| HDL (mg/dl) | 533 | 51.62 ± 12.85 | 424 | 62.69 ± 14.82 | 0.0001 |
| Log AER (mg/24 h) | 553 | 2.77 ± 1.46 | 446 | 2.48 ± 1.26 | 0.0011 |
| Common IMT(mm) | 520 | 0.6015 ± 0.0900 | 415 | 0.5620 ± 0.0761 | 0.0001 |
| Internal IMI (mm) | 511 | 0.6740 ± 0.2433 | 401 | 0.6148 ± 0.1842 | 0.0002 |
| % hypertensive | 554 | 0.44 ± 0.49 | 442 | 0.26 ± 0.44 | 0.0001 |
| % ACE Inhibitors | 562 | 0.14 ± 0.35 | 442 | 0.10 ± 0.30 | 0.0325 |

*variables evaluated between groups using t- and chi-square test for continuous and categorical variables, respectively.

TABLE 2

| | 95% CI | | | |
|---|---|---|---|---|
| Independent Variables | Effect | Lower | Upper | P |

Plasma

| | | | | |
|---|---|---|---|---|
| Age | 0.0129 | 0.0070 | 0.0188 | 0.0001 |
| Weight (kg) | 0.0010 | −0.0015 | 0.0035 | 0.4533 |
| BMI (kg/m²) | 0.0034 | −0.0068 | 0.0136 | 0.5145 |
| Waist-Hip Ratio | 0.3557 | −0.1325 | 0.8439 | 0.1513 |
| Duration of Diabetes (years) | 0.0124 | −0.0038 | 0.0210 | 0.0047 |
| HbAlc (%) | −0.0170 | −0.0480 | 0.0140 | 0.2826 |
| DCCT Group | 0.1031 | 0.0208 | 0.1853 | 0.0141 |
| SBP (mmHg) | 0.0039 | 0.0010 | 0.0069 | 0.0091 |
| DBF (mmHg) | 0.0005 | −0.0040 | 0.0049 | 0.8393 |
| Log AER(mg/24 h) | 0.0779 | 0.0481 | 0.1077 | 0.0000 |
| LDL | 0.0016 | 0.0002 | 0.0029 | 0.0270 |
| Total Cholesterol | 0.0016 | 0.0004 | 0.0028 | 0.0079 |
| Triglycerides | 0.0006 | 0.0000 | 0.0013 | 0.0672 |
| HDL | 0.0000 | −0.0028 | 0.0029 | 0.9851 |
| Internal Carotid MT | 0.2767 | 0.0811 | 0.4723 | 0.0056 |
| Common Carotid IMT | 1.1659 | 0.6744 | 1.6573 | 0.0000 |
| Gender % (male) | −0.0374 | −0.1203 | 0.2704 | 0.3765 |
| ACE Inhibitors | 0.1888 | 0.0643 | 0.3133 | 0.0030 |

URINE

| | | | | |
|---|---|---|---|---|
| Age | −0.0044 | −0.0113 | 0.0026 | 0.2166 |
| Weight (kg) | 0.0037 | 0.0008 | 0.0067 | 0.0129 |
| BMI (kg/m²) | 0.0050 | −0.0066 | 0.0167 | 0.3968 |
| Waist-Hip Ratio | 1.0875 | 0.5222 | 1.6527 | 0.0002 |
| Duration of Diabetes (years) | −0.0075 | −0.0175 | 0.0024 | 0.1392 |
| HbAlc (%) | 0.0061 | −0.0296 | 0.0417 | 0.7378 |
| DCCT Group | 0.0420 | −0.0538 | 0.1379 | 0.3899 |
| SBP (mmHg) | 0.0065 | 0.0030 | 0.0099 | 0.0002 |
| DBP (mmHg) | 0.0107 | 0.0056 | 0.0158 | 0.0001 |
| Log AER (mg/24 h) | 0.0033 | −0.0316 | 0.0382 | 0.8527 |
| LDL | −0.0004 | −0.0019 | 0.0012 | 0.6586 |
| Total Cholesterol | −0.0004 | −0.0018 | 0.0010 | 0.5923 |
| Triglycerides | 0.0003 | −0.0005 | 0.0011 | 0.4188 |
| HDL | −0.0015 | −0.0048 | 0.0018 | 0.3714 |
| Internal Carotid IMT | 0.1427 | −0.0836 | 0.3690 | 0.4146 |
| Common Carotid IMT | 0.2333 | −0.3395 | 0.8060 | 0.4243 |
| Gender (male) | −0.2091 | −0.3045 | −0.1137 | 0.0001 |
| ACE Inhibitors | 0.0550 | −0.0882 | 0.1982 | 0.4442 |

Univariable analysis showed a strong positive association between plasma log CTGF N-fragment levels and age, duration of diabetes, DCCT intensive group, systolic blood pressure, log AER, LDL, total cholesterol, internal carotid IMT, common carotid IMT, and the use of ACE-inhibitors. No association was observed between plasma log CTGF N fragment and HbA1c, gender, weight, body mass index, and waist-hip ratio.

A strong association between log urine CTGF N-fragment and weight, waist-hip ratio, SBP, DBP, and gender was observed. The excretion rate of log CTGF N-fragment was not influenced by age, duration of diabetes, HbA1c, log AER, total cholesterol, LDL, and ACEI.

### Example 3

### Association between Plasma CTGF N-fragment and Blood Pressure

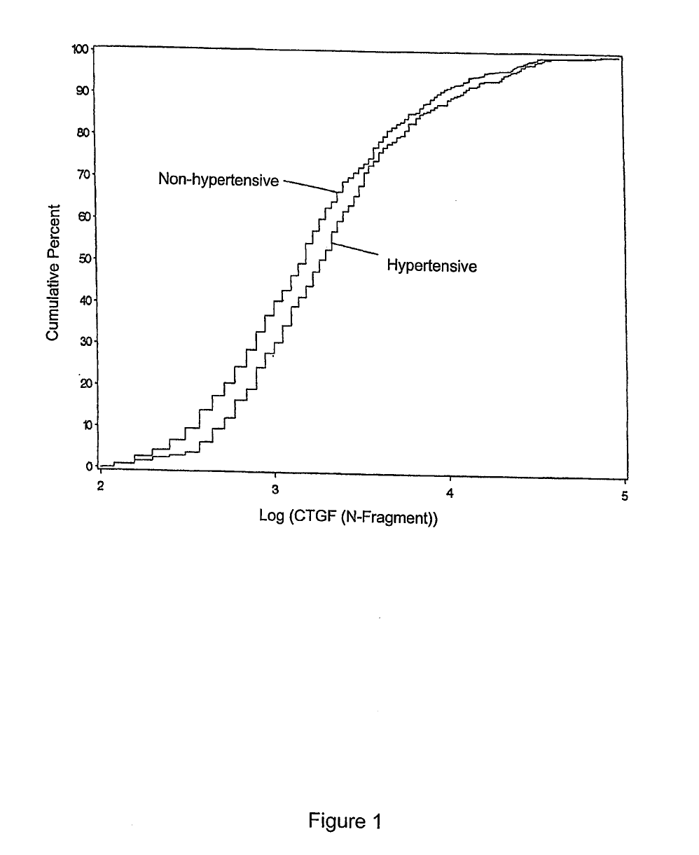

The results displayed in FIG. 1 show the estimated cumulative distribution of plasma log CTGF N-fragment plotted for non-hypertensive (n=668) and hypertensive (n=382) subjects (all patients diagnosed with hypertension, SBP>140, DBP>90; and treated with anti-hypertensive medication). These results demonstrate that hypertensive subjects tend to have higher values of plasma log CTGF N-fragment than do normotensive subjects.

The mean values of plasma log CTGF N-fragment for patients with documented hypertension is significantly greater than those for patients who did not develop hypertension (3.36±0.04 ng/ml vs. 3.21±0.03 ng/ml, P=0.0005). The actual mean plasma CTGF level measured in plasma of hypertensive patients is 41.57±3.47 ng/ml compared to an actual mean plasma CTGF level of 32.28±1.81 ng/ml in normotensive patients, P=0.0109. A strong association was also observed between the urinary excretion rate of log CTGF N-fragment and SBP, DBP, and MBP (all P<0.0001). These results demonstrate that plasma CTGF N-fragment levels are elevated in type 1 diabetic patients with hypertension.

Some of the hypertensive patients were on ACE-inhibitor (ACEI) therapy, and the influence, if any, of ACEI on the level of plasma and urine CTGF N-fragment levels in this patient cohort was examined. The results demonstrated that the mean plasma log CTGF N-fragment levels in patients treated with ACEI, 3.43±0.07 ng/ml (n=126), differed from a mean plasma log CTGF N-fragment level of 3.25±0.02 ng/ml (n=984) in patients not treated with ACEI, P=0.0159. On the other hand, ACEI therapy did not significantly influence the urinary excretion rate of log CTGF N-fragment. The mean urine log CTGF N-fragment in patients treated with ACEI was 2.13±0.07 μg/24 h (n=122) compared to a mean urine log CTGF N-fragment of 2.08±0.02 μg/24 h (n=906) in patients not treated with ACEI, P=0.4344.

### Example 4

### Relationship between CTGF N-fragment and Albumin Excretion

Plasma and urinary CTGF levels were measured in 1,052 type 1 diabetic patients and the results expressed as mean±SE are shown in Table 3 (plasma and urine CTGF N fragment levels by albuminuria status; P-values are compared to baseline group (AER<40) and are adjusted for age).

TABLE 3

| | AER < 40 mg/24 h | | AER 40-299 mg/24 h | | AER > 300 mg/24 h | |
|---|---|---|---|---|---|---|
| | (n = 896) | | (n = 105) | | (n = 51) | |
| Variable | Mean | SD | Mean | SD | Mean | SD |

Plasma (ng/ml)

| Log CTGF N | 3.227 | 0.657 | 3.297 | 0.542 | 3.750 | 0.921 |
|---|---|---|---|---|---|---|
| | | | (P = 0.2487) | | (P < 0.0001) | |
| CTGF N | 34.191 | 55.116 | 32.386 | 27.958 | 65.792 | 84.842 |

| | |
|---|---|
| (P = 0.8250) | (P = 0.0001) |

Urine (pg/24 h)

| Log CTGF N | 2.098 | 0.719 | 1.943 | 0.821 | 2.285 | 1.083 |
|---|---|---|---|---|---|---|
| | | | (P = 0.0529) | | (P = 0.1096) | |
| CTGF N | 10.611 | 10.142 | 9.248 | 7.189 | 18.534 | 30.918 |

| | |
|---|---|
| (P = 0.2806) | (P < 0.0001) |

The data demonstrated that CTGF N-fragment levels in plasma and urine of patients with macroalbuminuria (albumin excretion rate >300 mg/dl) were significantly higher than those in plasma and urine of patients with microalbuminuria (40-299 mg/dl) or patients with normal albumin excretion rate (<40 mg/dl), consistent with previous reports showing a correlation between the level of CTGF N-fragment in urine and the degree of albuminuria. (See International Publication No. WO 03/024308.) Plasma CTGF N-fragment levels in patients with AER ≧300 mg/dl were 65.79±11.89 ng/ml vs. 32.39±2.73 ng/ml in patients with AER of 40-299 ng/dl and 34.19±1.84 ng/ml in patients with normal AER <40 mg/dl (P=0.0005), consistent with previous reports showing a correlation between the level of CTGF N-fragment in urine and the albumin excretion rate. (See International Publication No. WO 03/024308.) The excretion rate of CTGF N-fragment in patients with AER≧300 mg/dl was 18.53±4.33 μg/24 h vs. 9.25±0.70 μg/24 h in patients with AER of 40-299 mg/dl and 10.61±0.34 μg/24 h in patients with normal AER<40 mg/dl; P=0.0001. These findings suggested that CTGF serves as a diagnostic marker that effectively identifies patients with an increased risk of progression to macroalbuminuria.

Table 2 showed a positive and significant association between plasma log CTGF N-fragment and log albumin excretion rate (P=0.001, n=1052). The strength of the association of plasma log CTGF N-fragment with log AER was further evaluated by multiple linear regression analyses. A multiple regression model was developed based on the univariate regression analysis used for the data shown in Table 2, but with log AER as the outcome rather than plasma log CTGF N fragment. A number of variables that may influence log AER were included in this model. Non-significant variables and co-linear variables were eliminated by backward regression analysis to develop a model that best explained the outcome as a function of the diagnostic marker indicative of increased risks. The results shown in Table 4 (multiple linear regression models for log AER) demonstrated, after controlling for age, weight, BMI, duration of diabetes, HbAlc, DCCT intensive group, SBP, and total cholesterol, a significant association between plasma log CTGF and log AER (P<0.0001). These results were interpreted to show that a two-fold difference in plasma CTGF N-fragment resulted in a 20% increase in AER.

TABLE 4

| | 95% CI | | | |
|---|---|---|---|---|
| Variable | Effect | Lower | Upper | P |
| Intercept | −4.3388 | −5.5492 | −3.1824 | 0.0001 |
| Plasma log CTGF N fragment | 0.3183 | 0.1959 | 0.4407 | 0.0001 |
| Age | −0.0272 | −0.0393 | −0.0150 | 0.0001 |
| Weight (kg) | 0.0008 | 0.0002 | 0.0161 | 0.0445 |
| BMI (kglm²) | −0.0348 | −0.0645 | −0.0050 | 0.0221 |
| Waist-Hip ratio | 0.2832 | −0.8149 | 1.3859 | 0.6143 |
| Duration of diabetes (years) | 0.0388 | 0.0219 | 0.0557 | 0.0001 |
| HbAlc(%) | 0.2414 | 0.1797 | 0.3026 | 0.0001 |
| DCCT intensive group | 0.3792 | 0.2179 | 0.5405 | 0.0001 |
| SBP (mmHg) | 0.0240 | 0.0179 | 0.0301 | 0.0001 |
| Total Cholesterol (mg/dl) | 0.0052 | 0.0028 | 0.0076 | 0.0001 |

### Example 5

### CTGF and Carotid Arterial Wall Thickness

The relationship between CTGF activity and common and/or internal carotid intima-media thickness (IMT) was examined to determine whether differences in plasma CTGF N-fragment levels were associated with macrovascular disease. Univariate analysis demonstrated significant association between plasma log CTGF N fragment levels and the common and internal carotid IMT (both P<0.0001) in 1,050 participants (Table 2). These findings indicated that plasma CTGF levels are positively related to carotid IMT, a surrogate marker for macrovascular disease.

A multiple regression model was constructed to assess the strength of the association of plasma log CTGF N-fragment and common carotid IMT. Performing backward regression analysis, the model containing plasma log CTGF N-fragment, log AER, age, and gender (Table 5, multiple linear regression models for common carotid IMT) demonstrated that log CTGF N-fragment independently and significantly associate with the common carotid IMT.

TABLE 5

| | 95% CI | | | |
|---|---|---|---|---|
| Variable | Effect | Lower | Upper | P |
| Intercept | 0.3981 | 0.3578 | 0.4384 | 0.0001 |
| Plasma log CTGF N fragment | 0.0079 | 0.0003 | 0.0156 | 0.0428 |
| Log AER | 0.0112 | 0.0075 | 0.0149 | 0.0001 |
| Age | 0.0045 | 0.0037 | 0.0052 | 0.0001 |
| Gender | −0.0319 | −0.0420 | −0.0218 | 0.0001 |

IMT was dichotomized into high and low categories based on the 75^{th} percentile and fitted into a logistic regression model with this as the outcome. Logistic models adjusted for age confirmed the association of CTGF with increased risk carotid IMT. The results shown demonstrated that subjects with high CTGF levels and AER≧300 mg/day have a significantly greater risk for increased carotid IMT (relative risk: 4.76; 95% CI, 2.21-10.25, P<0.0001) than do subjects with low CTGF levels and AER<40 mg/day. (See Table 6, relative risk for increased carotid IMT@ according to plasma CTGF N fragment and AER.)

TABLE 6

| | | AER < 40 mg/day | AER > 40 < 299 mg/day | AER > 300 mg/day |
|---|---|---|---|---|
| CTGF N | High | RR = 1.86 (1.30-2.67) | RR = 2.54 (1.21-5.23) | RR = 4.76 (2.21-10.25) |
| | | n = 376 (P = 0.0007) | n = 41 (P = 0.0135) | n = 36 (P < 0.0001) |
| | Low | RR = 1.00 | RR = 2.42 (1.13-5.18) | RR = 2.69(0.59-12.92) |
| | | n = 415 | n = 43 (P = 0.0224) | n = 9 (P = 0.1989) |
| | Total | n = 791 | n = 84 | n = 45 |

RR = Relative Risk (95% confidence interval)
^{@} Increased IMT defined as top quartile of distribution for men and women combined
** Estimates are age adjusted

As described above, the present study is based on data generated from the DCCT/EDIC cohort of type 1 diabetic patients and indicates that diabetic vascular disease is linked to abnormalities CTGF levels. These findings provide evidence of an independent and positive association between CTGF N-fragment levels and surrogate markers of macrovascular disease (common and carotid IMT). In addition, the present findings demonstrate an independent association between CTGF N-fragment levels and hypertension and microalbuminuria, both of which are diagnostic markers indicative of increased risks for the development of macrovascular disease. Furthermore, these cross-sectional results demonstrate that the relative risk for carotid IMT is increased in diabetic subjects with high CTGF levels.

What is claimed is:

1. A method for diagnosing a risk for development of a vascular complication associated with diabetes in a subject having or at risk for having diabetes, the method comprising obtaining a biological sample from the subject, measuring the level of CTGF or of CTGF fragment in that biological sample, and comparing the level of CTGF or of CTGF fragment in the biological sample to standard levels of CTGF or of CTGF fragment, where an elevated level of CTGF or of CTGF fragment in the biological sample is indicative of a risk for development of a vascular complication associated with diabetes.

2. The method of claim 1, wherein the subject having or at risk for having diabetes is a human subject.

3. The method according to claim 1 or claim 2, wherein the vascular complication is a macrovascular complication or a microvascular complication.

4. The method according to any of the preceding claims, wherein the vascular complication is selected from the group consisting of a cardiovascular complication, a cerebrovascular complication, and a complication of the peripheral vasculature.

5. The method according to any of the preceding claims, wherein the vascular complication is increased intima-medial thickness.

6. The method according to any of the preceding claims, wherein the level of CTGF fragment or of CTGF in the biological sample is detectable and quantifiable using an assay described in International Publication No. WO 03/024308.

7. The method according to any of the preceding claims, wherein the biological sample is a sample derived from bodily fluids.

8. The method according to any of the preceding claims, wherein the biological sample is urine or plasma.

9. The method according to any of the preceding claims, wherein the subject has type 1 diabetes.

10. The method according to any of the preceding claims, wherein the subject has increased blood pressure.

11. The method according to any of the preceding claims, wherein the subject has microalbuminuria.
